(19) United States
(12) Patent Application Publication
(10) Pub. No.: US 2023/0282320 A1
(43) Pub. Date: Sep. 7, 2023

(54) GENERATIVE MOTION MODELING USING EXTERNAL AND INTERNAL ANATOMY INFORMATION

(71) Applicant: Varian Medical Systems International AG, Steinhausen (CH)

(72) Inventors: Ricky R. Savjani, Santa Clara, CA (US); Pascal Paysan, Basel (CH); Stefan Georg Scheib, Waedenswil (CH)

(73) Assignee: Varian Medical Systems International AG, Steinhausen (CH)

(21) Appl. No.: 17/688,542

(22) Filed: Mar. 7, 2022

(51) Int. Cl. G16H 10/60; G16H 30/20; G06N 3/08; A61N 5/10

(52) U.S. Cl. CPC G16H 10/60; G16H 30/20; G06N 3/08; A61N 5/1048

(57) ABSTRACT

Provided herein are methods and systems to train and execute a motion model that uses artificial intelligence methodologies (e.g., deep-learning) to learn and predict location of a patient's internal structures. A method comprises receiving respiratory data of a patient from an electronic sensor in addition to a medical image, such as kV image; executing an artificial intelligence model using the respiratory data and predicting deformation data for at least one internal structure of the patient, wherein the artificial intelligence model is trained in accordance with a training dataset comprising a set of participants, their corresponding respiratory data, and their corresponding deformation data; and outputting the predicted deformation data.

### TECHNICAL FIELD

This application relates generally to using data analysis techniques to model and predict patient attributes during radiotherapy treatment and to control a radiotherapy machine.

### BACKGROUND

One of the major challenges in image-guided radiation therapy (IGRT) is addressing various types of patient motion. The motion can be both cyclical motion (e.g., components of respiratory and cardiac motion) as well as irregular motion (e.g., gastrointestinal events including peristalsis, swallowing, and the passage of gas bubbles, muscle relaxation in breath-hold, and body and limb movement).

IGRT attempts to mitigate the effects of motion in many ways, but there are two major deficiencies with IGRT. First, no estimate of the delivered dose (as opposed to planned dose) is usually computed. Second, no real-time three-dimensional (3D) volumetric depiction of patient anatomy/motion is visualized during treatment. Although there are imaging techniques attempting to resolve motion with respect to the respiratory or cardiac cycle, conventional imaging devices and systems do not provide real-time resolved 3D information about the patient at every time instance. Moreover, conventional four-dimensional (4D) imaging techniques (e.g., positron emission tomography (PET), Magnetic resonance (MR) imaging, computerized tomography (CT), and/or cone beam computerized tomography (CBCT)) generally rely on a retrospective reconstruction of the data and are not real-time capable or proactive.

### SUMMARY

For the aforementioned reasons, there is a desire for a system that can rapidly and accurately analyze patient information and provide a projected location of a patient's internal structures. Using the methods and systems discussed herein, a computer model (e.g., artificial intelligence (AI) model) can account for patient movements. Using the methods and systems discussed herein, a processor can use deep learning to train intra-patient and inter-patient motion models. Applications of these computer models can be in the field of real-time tissue tracking during radiation beam delivery, real-time motion visualization, retrospective and/or real-time delivered dose calculation, organ/segmentation (e.g., gross tumor volume (GTV)), specific dose tracking, outcome prediction, and image reconstruction.

The methods and systems discussed herein (unlike classical tracking methods) may allow an AI model to learn the space of anatomical feasible deformation and therefore have the capability to infer the 3D anatomy from limited data (e.g., single kV projections or with proper initialization from a surrogate signal for respiration, such as the patient 3D surface, a real-time position management (RPM), or electrocardiogram (ECG) trace, stereoscopic kV imaging, kV+MV imaging and digital tomosyntheses (kV and MV), or combinations of those). AI models can be trained to analyze and predict patient motion (both external and internal). These AI models may be trained to use real-time inputs received from the patient and/or external sensors.

One or more AI models can be trained for the prediction of physiological valid deformations. In the field of radiotherapy, physiological valid deformations may be key to several applications, such as dose accumulation, structure propagation, and tracking of tissues with little to no contrast. The AI models discussed herein can be used to predict various deformations and the predicted results can be transmitted to one or more downstream applications where other software solutions can use the predicted data and calculate/predict other attributes needed to implement and perform the patient's treatment.

One or more AI models can learn and leverage correlations between moving tissues of a patient. By learning the relative motion of tissues (based on a population of patients within a training dataset and inferring data based on a particular patient being treated), an AI model may infer/predict local deformation from limited information. This information can be (but not limited to) one or a combination of surrogate signals for respiration or heartbeat (e.g. RPM, 3D surface, ECG), kV projections acquired (e.g. during treatment triggered or fluoroscopic images and/or tomosyntheses), detected high-contrast objects in projections such as metal markers, ultrasound or radar measurements, or other. The models can be either fitted to the data or correlated to the respective signals.

One or more AI models can incorporate and analyze data associated with patient physiological dynamics. Temporal analysis of biomechanics and its modeling can be used additionally to predict the patient anatomy.

One or more AI models can be trained as multimodal motion models. Since motion models encode possible deformations within the body, they may be, in principle, independent of the underlying imaging technology. This means that it becomes feasible to use image sequences from different sources jointly to build such models or adapt them for each patient individually.

One or more AI models can be trained to correspond to a common reference anatomy. The AI models can encode the deformation with respect to a certain reference anatomy that could be a defined patient-specific template (e.g., CT simulation) or an inter-subject patient atlas. This allows for novel intra- and inter-fraction dose accumulation, structure propagation, and outcome prediction methods derived from population statistics.

One or more AI models can constrain motion-compensated image reconstruction. Since motion models can provide prior information about possible deformations in the body in form of a probability, one can apply them during image reconstruction where deformations are predicted based on limited information.

A deep-learning-based motion model can be trained on motion resolved volumetric images across different patients. This model can then be used to predict the current deformation of a patient with respect to a volumetric static image of the same patient (e.g. planning CT or CBCT) based on one or more surrogate signals (e.g., RPM, ECG, 3D surface, one or more kV projections, digital tomosyntheses, which could also include stereoscopic imaging, and/or ultrasound). The AI model discussed herein can be applied to help with the patient's treatment in at least two different ways. First, the AI model can be applied during post-treatment verification. Second, the AI model can be applied during treatment for real or near real-time prediction. The prediction of the deformation can be used for visualization purposes, tracking of structures identified on the static image, deformation of planned dose (beamlets), and re-calculation of delivered dose. Particularly, the deformation-dependent dose calculation enables the tracking of PTV and organ at risk (OAR) dose with the goal to achieve dose constraints using the actual delivered dose.

The AI model can leverage data associated with a set of patients (data across patients or a cohort of patients) in combination with patient-specific information. This allows the model to be trained using a set of participants (e.g., previously treated patients). The AI model can then use the training to predict a deformation associated with a patient. The methods and systems discussed herein (e.g., using deep-learning to train the AI model) enables the combination of the patient-specific information in conjunction with training obtained from a cohort of patients. This overcomes the limitations (overfitting) of patient-specific models to represent novel patient motion states (not in the span of the model).

The model can learn temporal and biomechanical knowledge associated with the patient and the patient's cohort. Due to the high complexity of classical approaches to model biodynamics (e.g., finite elements), it has not been possible so far to utilize certain temporal and/or biomechanical attributes in clinical practice. Data-driven methods described herein can enable the use of temporal and biomechanical knowledge. The AI model can learn anatomical and physiological properties of a patient and/or the patient's cohorts, which was previously not possible/feasible (e.g., using the sequential application of deformable image registration (DIR) followed by modeling).

The usage of AI models that are trained in an unsupervised or semi supervised model has the potential to learn anatomical properties like sliding interfaces in contrast to classical approaches where the modeling is usually based on standard deformable image registration. In this way, physiological deformation of the bones can be prevented which makes the model applicable for dose calculation.

The trained AI model may also provide anatomically valid interpolations. To predict continuous anatomical deformation, it may be necessary to interpolate between discrete time-steps represented by the AI model (e.g., one can use piecewise linear interpolation between phases of a 4D CT/CBCT). The AI models discussed herein can utilize auto-encoders trained such that interpolation in the latent space yields anatomical valid intermediate steps.

In contrast to heuristic-designed or classically optimized methods, the AI model discussed herein can allow for much more complex loss/objective functions. This permits optimization of the model prediction directly with respect to the clinical application (e.g. dose calculation or segmentation). Therefore, the prediction provided by the AI model can be used as basis for downstream dose calculation software solutions. The AI model can be trained, such that it is an imaging-modality agnostic model. In addition to leveraging information across patients, it is also possible to train the AI model across different modalities. This allows the prediction of plausible deformation in regions where kV imaging does not show tissue contrast. As a result, the trained model can be applied for image reconstruction (due to the additional expressiveness).

In contrast to classical motion models (e.g., principal component analysis (PCA) based) deep-learning-based models can be nonlinear and may generalize better to novel anatomy. Therefore, the model can be used as guidance during (e.g., motion-compensated) reconstruction or treatment without introducing additional/inappropriate deformations.

The AI model can provide a prediction of physiological valid deformations. Conventional methods, such as DIR methods, map intensities onto each other while constraining the deformation with a smoothness term. This simple smoothness assumption is insufficient for various tasks in radiotherapy such as dose deformation and accumulation. This is because certain organs show uniform gray values for the given image modality and therefore the estimated deformation within the region is purely defined by the smoothness assumption. On the other hand, the smoothness assumption does not allow for discontinuities in the deformation at sliding interfaces such as between the ventral cavity and liver.

The AI models can be trained by applying advanced loss functions, model constraints, or using supervised approaches with known valid deformations. Loss functions and model constraints can be defined such that they account for tissue-dependent changes of deformability (e.g., rigid bones and/or compressible lung tissue) or the sliding interfaces. The model may the cyclic nature of periodic motion into account while the analytics server may constraint such data. For instance, features with high contrast but no meaningful correspondence as moving air cavities can be ignored. Knowing the deformation of the patient anatomy in such a manner allows for dose accumulation on reference anatomy. For instance, the physiological constraint deformation may allow avoiding artifacts in the dose.

The AI model may allow for accumulation due to incorrect deformations. The accumulation of deformed doses can be applied for deformation sequences during or after treatment. This allows for dose monitoring of different structures during treatment and take action (e.g., beam hold) or after beam delivery to adapt or validate/record the treatment.

The AI model may also allow outcome prediction by mapping applied dose to a single atlas either per patient or across patients and making inferences with data collected during follow-up. The AI model may also use a-priori knowledge for motion compensation and/or image reconstruction.

The AI model can provide learning and leverage correlations between moving tissue and surrogate signals. Certain conventional systems (e.g., conventional standard of care using motion management) assume a strong correlation between the tumor and a surrogate signal such as the RPM signal or a 3D surface. Since the AI models discussed herein can predict patient anatomy (in particular correlations between moving tissues) it can be enabled apart from the prediction of the GTV motion relative to the surrogate e.g., the position estimation of OARs (allowing motion management strategies to minimize the dose to healthy tissue).

Because the AI model discussed herein can encode the correlation between any structure in the body it could be applied to improve tracking by constraining (e.g., the detected tumor position based on detected landmarks). For instance, the AI model may identify that a tumor can only move tangential to the rib cage or nearly coherent with the diaphragm or markers. The modeling of the anatomical site enables also the usage of a combination of surrogates like kV projections, ECG, RPM, 3D surface, radar, or ultrasound. For instance, in non-coplanar beam incidences, the model and its predictions can temporarily prevent (or provide the option for the medical professional to stop) imaging during treatment, the model can predict patient anatomy change solely based on surrogates. The known correlation between points can as well be used during deformable image registration to constrain possible solutions (e.g. within organs, at sliding interfaces, or rigid bones).

The AI model can provide data-driven incorporation of physiological dynamics. In some embodiments, the AI model can learn dynamic processes from the training data. The AI model may learn anatomy-dependent constraints on acceleration. Furthermore, the AI model discussed herein may utilize recurrent networks such as LSTM models to learn temporal dependent/sequential or periodic aspects.

The AI model can provide multimodal motion services. The AI model may also be trained to transform images to other modalities. This technique could be used to bring images of different modalities into correspondence and use them for the model building based on different modalities, such as 4D MM and 3D/4D CT. Moreover, the analytics server may train a predictive deformable registration algorithm agnostic to the modality. This could be done based on a suitable similarity metric that can compare the different modalities, based on image pairs that have the same geometry/anatomy such that the correspondence is known, or based on simulated images of different modalities.

The AI model can be generated and trained with respect to one (or more) common reference anatomy/atlas. Population-based models have the ability to factorize patient images into anatomical appearance (e.g., patient-specific tissue and/or fillings of the gastrointestinal) and shape. The AI model can use this technique to compare images (patient images and/or predicted or reconstructed images) with a reference (e.g., common reference image). Using this technique the AI model may establish comparability between different subjects and therefore statistically evaluate them (e.g., distribution of lung tumors with respect to clinical outcome).

The AI model may also be used to constrain motion-compensated image reconstruction. One way to improve image quality in image reconstruction is to estimate the motion that took place throughout the image acquisition and compensate for it (e.g., by warping to a common reference). Since the deformations need to be estimated additionally to the image content the already ill-posed reconstruction becomes more challenging. Using classical optimization algorithms as iterative reconstruction or deformable image registration means that the complexity of such constraints is limited to allow for convergence. Finite element methods introduce more precise physiological models but are computationally intensive and difficult to implement. The AI model may be trained to learn the plausibility of deformations in form of probabilistic models capturing prior information. This prior information can then be used throughout the reconstruction process to constrain the space of possible solutions. This can allow making a valid prediction of the deformation based on the limited data (e.g., because of time or dose constraints) captured throughout the acquisition.

In an embodiment, a method comprise receiving, by a processor, respiratory data of a patient from an electronic sensor; executing, by the processor, an artificial intelligence model using the respiratory data and predicting deformation data for at least one internal structure of the patient, wherein the artificial intelligence model is trained in accordance with a training dataset comprising a set of participants, their corresponding respiratory data, and their corresponding deformation data; and outputting, by the processor, the predicted deformation data.

The method may further comprise receiving, by the processor, a medical image of the patient, wherein the processor executes the artificial intelligence model using the medical image.

The respiratory data received from the electronic sensor may be at least one of a chest position, chest movement, or respiratory cycle data of the patient.

The deformation data may correspond to a movement of at least one internal structure of the patient.

The method may further comprise adjusting, by the processor, at least one attribute of a radiotherapy machine in accordance with the predicted deformation data.

The at least one attribute may correspond to a multi-leaf collimator opening.

Outputting the predicted deformation data may correspond to a simulated medical image depicting an anatomical region of the patient.

Outputting the predicted deformation data may correspond to transmitting the predicted deformation data to a dose calculation software solution or a tissue tracking software solution.

The artificial intelligence model may generate predicted respiratory data associated with the patient, the predicted respiratory data comprising at least one of a chest movement or an attribute of a respiratory cycle.

The electronic sensor may be a wearable respiratory sensor or an optical respiratory sensor.

In another embodiment, a computer system comprises a server comprising a processor and a non-transitory computer-readable medium containing instructions that when executed by the processor causes the processor to perform operations comprising: receiving respiratory data of a patient from an electronic sensor; executing an artificial intelligence model using the respiratory data and predicting deformation data for at least one internal structure of the patient, wherein the artificial intelligence model is trained in accordance with a training dataset comprising a set of participants, their corresponding respiratory data, and their corresponding deformation data; and outputting the predicted deformation data.

The instructions may further cause the processor to receive a medical image of the patient, wherein the processor executes the artificial intelligence model using the medical image.

The respiratory data received from the electronic sensor may be at least one of a chest position, chest movement, or respiratory cycle data of the patient.

The deformation data may correspond to a movement of at least one internal structure of the patient.

The instructions may further cause the processor to adjust at least one attribute of a radiotherapy machine in accordance with the predicted deformation data.

The at least one attribute may correspond to a multi-leaf collimator opening.

Outputting the predicted deformation data corresponds to a simulated medical image depicting an anatomical region of the patient.

Outputting the predicted deformation data corresponds to transmitting the predicted deformation data to a dose calculation software solution or a tissue tracking software solution.

The artificial intelligence model may generate predicted respiratory data associated with the patient, the predicted respiratory data comprising at least one of a chest movement or an attribute of a respiratory cycle.

The electronic sensor may be a wearable respiratory sensor or an optical respiratory sensor.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates components of an artificial intelligence motion modeling system, according to an embodiment.

FIG. 2 illustrates a process flow diagram of an artificial intelligence motion modeling system, according to an embodiment.

FIG. 3 illustrates a visual representation of respiration data for a set of patients, in accordance with an embodiment.

FIG. 4 illustrates a visual representation of respiration data and corresponding medical images depicting movement of one or more internal structures, in accordance with an embodiment.

FIG. 5 illustrates a visual representation of a vectorized medical image, in accordance with an embodiment.

FIG. 6 illustrates a visual representation of training an AI model, in accordance with an embodiment.

FIG. 7 illustrates a visual representation of medical images analyzed and generated by an AI model, in accordance with an embodiment.

FIG. 8 illustrates a visual representation of deformation vectors, in accordance with an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used and/or other changes may be made without departing from the spirit or scope of the present disclosure. The illustrative embodiments described in the detailed description are not meant to be limiting of the subject matter presented.

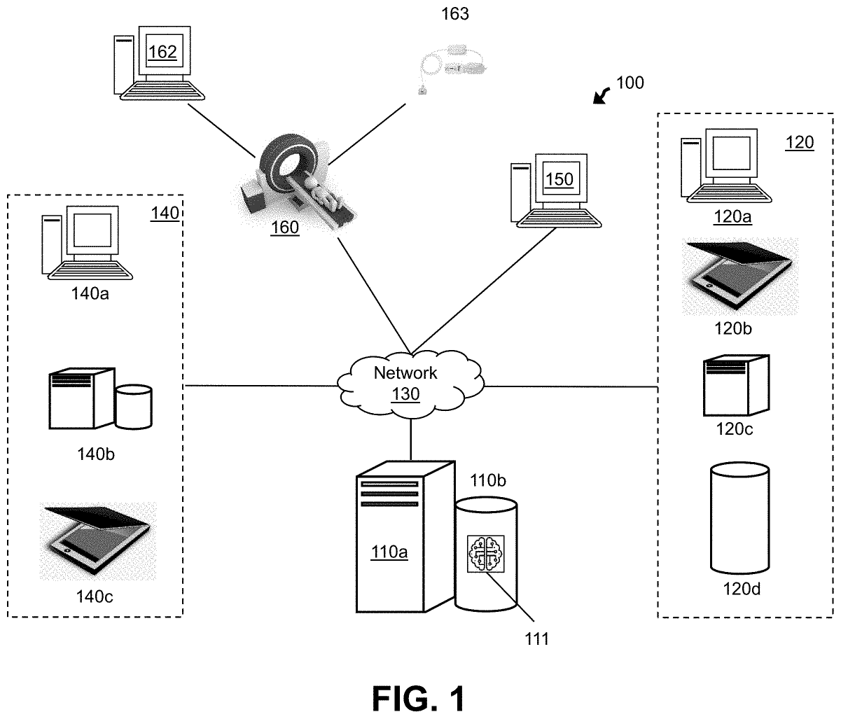

FIG. 1 illustrates components of a system 100 for an artificial intelligence motion modeling system, according to an embodiment. The system 100 may include an analytics server 110a, system database 110b, an AI model 111, electronic data sources 120a-d (collectively electronic data sources 120), end-user devices 140a-c (collectively end-user devices 140), an administrator computing device 150, and medical device 160, medical device computer(s) 162, and a respiration sensor 163. Various components depicted in FIG. 1 may belong to a radiotherapy clinic at which patients may receive radiotherapy treatment, in some cases via one or more radiotherapy machines located within the clinic (e.g., medical device 160). Additionally or alternatively, the AI model 111 can be implemented using any 4D image, e.g. 4D-MRI which has been acquired for any other use as well, which may not be connected to radiation therapy.

The system 100 is not confined to the components described herein and may include additional or other components, not shown for brevity, which are to be considered within the scope of the embodiments described herein.

The above-mentioned components may be connected to each other through a network 130. Examples of the network 130 may include, but are not limited to, private or public local-area-networks (LAN), wireless LAN (WLAN) networks, metropolitan area networks (MAN), wide-area networks (WAN), and the Internet. The network 130 may include wired and/or wireless communications according to one or more standards and/or via one or more transport mediums. The communication over the network 130 may be performed in accordance with various communication protocols such as Transmission Control Protocol and Internet Protocol (TCP/IP), User Datagram Protocol (UDP), and IEEE communication protocols. In one example, the network 130 may include wireless communications according to Bluetooth specification sets or another standard or proprietary wireless communication protocol. In another example, the network 130 may also include communications over a cellular network, including, e.g., a GSM (Global System for Mobile Communications), CDMA (Code Division Multiple Access), EDGE (Enhanced Data for Global Evolution) network.

The analytics server 110a may generate and display an electronic platform configured to use various AI models 111 (including artificial intelligence and/or machine learning models) for receiving patient information and outputting the results of execution of the AI models 111. The electronic platform may include graphical user interfaces (GUI) displayed on each electronic data source 120, the end-user devices 140, the medical device 160, and/or the administrator computing device 150. An example of the electronic platform generated and hosted by the analytics server 110a may be a web-based application or a website configured to be displayed on different electronic devices, such as mobile devices, tablets, personal computers, and the like.

The information displayed by the electronic platform can include, for example, input elements to receive data associated with a patient being treated, synchronize one or more sensors, such as the patient sensor 163, and display results of predictions produced by the AI model 111 (e.g., a reconstructed image for the patient that displays location of a tumor predicted by the AI 111). For instance, the analytics server 110a may execute the AI model 111 (e.g., machine learning models trained to generate predicted tumor locations and/or breathing patterns for a patient being treated via the medical device 160). The analytics server 110a may then display the results for a medical professional and/or directly revise one or more operational attributes of the medical device 160. In some embodiments, the medical device 160 can be a diagnostic imaging devices or a treatment delivery device.

The analytics server 110a may be any computing device comprising a processor and non-transitory machine-readable storage capable of executing the various tasks and processes described herein. The analytics server 110a may employ various processors such as central processing units (CPU) and graphics processing unit (GPU), among others. Non-limiting examples of such computing devices may include workstation computers, laptop computers, server computers, and the like. While the system 100 includes a single analytics server 110a, the analytics server 110a may include any number of computing devices operating in a distributed computing environment, such as a cloud environment.

The electronic data sources 120 may represent various electronic data sources that contain, retrieve, and/or access data associated with a medical device 160, such as operational information associated with previously performed radiotherapy treatments (e.g., electronic log files or electronic configuration files), data associated with previously monitored patients (e.g., breathing patterns, tumor location, deformation information) or participants in a study to train the AI models discussed herein. For instance, the analytics server 110a may use the clinic computer 120a, medical professional device 120b, server 120c (associated with a physician and/or clinic), and database 120d (associated with the physician and/or the clinic) to retrieve/receive data associated with the medical device 160. The analytics server 110a may retrieve the data from the end-user devices 120, generate a training dataset, and train the AI models 111. The analytics server 110a may execute various algorithms to translate raw data received/retrieved from the electronic data sources 120 into machine-readable objects that can be stored and processed by other analytical processes as described herein.

End-user devices 140 may be any computing device comprising a processor and a non-transitory machine-readable storage medium capable of performing the various tasks and processes described herein. Non-limiting examples of an end-user device 140 may be a workstation computer, laptop computer, tablet computer, and server computer. In operation, various users may use end-user devices 140 to access the GUI operationally managed by the analytics server 110a. Specifically, the end-user devices 140 may include clinic computer 140a, clinic server 140b, and a medical processional device 140c. Even though referred to herein as “end-user” devices, these devices may not always be operated by end-users. For instance, the clinic server 140b may not be directly used by an end user. However, the results stored onto the clinic server 140b may be used to populate various GUIs accessed by an end user via the medical professional device 140c.

The administrator computing device 150 may represent a computing device operated by a system administrator. The administrator computing device 150 may be configured to display radiotherapy treatment attributes generated by the analytics server 110a (e.g., various analytic metrics determined during training of one or more machine learning models and/or systems); monitor various models 111 utilized by the analytics server 110a, electronic data sources 120, and/or end-user devices 140; review feedback; and/or facilitate training or retraining (calibration) of the AI model 111 that are maintained by the analytics server 110a.

The medical device 160 may be a radiotherapy machine configured to implement a patient's radiotherapy treatment. The medical device 160 may also include an imaging device capable of emitting radiation such that the medical device 160 may perform imaging according to various methods to accurately image the internal structure of a patient. For instance, the medical device 160 may include a rotating system (e.g., a static or rotating multi-view system). A non-limiting example of a multi-view system may include stereo systems (e.g., two systems may be arranged orthogonally). The medical device 160 may also be in communication with a medical device computer 162 that is configured to display various GUIs discussed herein. For instance, the analytics server 110a may display the results predicted by the AI model 111 onto the computing devices described herein.

The medical device 160 may also include one or more sensors configured to monitor the patient being treated. For instance, the medical device 160 may include 3D surfacing mechanisms and sensors (e.g., optical sensors) configured to monitor the patient's movements (e.g., how the patient is moving and/or breathing). In some embodiments, the medical device 160 may be in communication with a respiratory sensor 163. The respiratory sensor 163 may be any sensor configured to monitor the patient's breathing. For instance, the respiratory sensor may be a strap configured to monitor the patient's chest position and movement, whereby a processor (e.g., internal to the respiratory sensor 163 or the analytics server 110a) can analyze to identify how the patient is breathing. Data received from the respiratory sensor 163 may also be referred to as the surrogate signal.

The AI model 111 may be stored in the system database 110b. The AI model 111 may be trained using data received/retrieved from the electronic data sources 120 and may be executed using data received from the end-user devices, the medical device 160, and/or and the sensor 163. In some embodiments, the AI model 111 may reside within a data repository local or specific to a clinic. In various embodiments, the AI models 111 use one or more deep learning engines to generate a predicted breathing patterns and organ deformity for a patient being treated. For instance, the analytics server 110a may transmit patient attributes from the sensor 163 and execute the AI models 111 accordingly.

It should be understood that any alternative and/or additional machine learning model(s) may be used to implement similar learning engines. The deep learning engines can include processing pathways that are trained during a training phase. Once trained, deep learning engines may be executed (e.g., by the analytics server 110a) to generate predicted patient attributes.

As described herein, the analytics server 110a may store the AI model 111 (e.g., neural networks, random forest, support vector machines, regression models, recurrent models, etc.) in an accessible data repository. The analytics server 110a may retrieve the AI models 111 and train the AI models 111 to predict a deformity associated with one or more of the patient's structures/organs.

Various machine learning techniques may involve “training” the machine learning models to predict (e.g., estimate the likelihood of) patient attributes, including supervised learning techniques, unsupervised learning techniques, or semi-supervised learning techniques, among others. In a non-limiting example, the predicted patient attribute may indicate a patient's predicted breathing pattern and deformity of the patient's structure (e.g., tumor). The AI model 111 can therefore be used to predict a real-time location and orientation of the PTV. As a result, the analytics server 110a may display the tumor's projected location and/or revise the patient's treatment accordingly, such as by changing the MLC openings.

One type of deep learning engine is a deep neural network (DNN). A DNN is a branch of neural networks and consists of a stack of layers each performing a specific operation, e.g., convolution, pooling, loss calculation, etc. Each intermediate layer receives the output of the previous layer as its input. The beginning layer is an input layer, which is directly connected to or receives an input data structure that includes the data items in one or more machine-readable objects, and may have a number of neurons equal to the data items in one or more machine-readable objects provided as input. For example, a machine-readable object may be a data structure, such as a list or vector, which includes a number of data fields include data received from the sensor 163. Each neuron in an input layer can accept the contents of one data field as input. The analytics server 110a may pre-process the machine-readable objects (e.g., through an encoding process) such that the data fields may be accepted as input to the AI model 111 described herein.

A next set of layers can include any type of layer that may be present in a DNN, such as a convolutional layer, a fully connected layer, a pooling layer, or an activation layer, among others. Some layers, such as convolutional neural network layers, may include one or more filters. The filters, commonly known as kernels, are of arbitrary sizes defined by designers. Each neuron can respond only to a specific area of the previous layer, called receptive field. The output of each convolution layer can be considered as an activation map, which highlights the effect of applying a specific filter on the input. Convolutional layers may be followed by activation layers to apply non-linearity to the outputs of each layer. The next layer can be a pooling layer that helps to reduce the dimensionality of the convolution's output. In various implementations, high-level abstractions are extracted by fully connected layers. The weights of neural connections and the kernels may be continuously optimized in the training phase.

In practice, training data may be user-generated through observations and experience to facilitate supervised learning. For example, training data may be received and monitored during past radiotherapy treatments provided to prior patients. In another example, the training data may be a dataset that includes breathing patterns of patient while being treated and their corresponding movements (e.g., chest position and movement of patients and their breathing patters and their corresponding timestamped medical images). Training data may be pre-processed via any suitable data augmentation approach (e.g., normalization, encoding, any combination thereof, etc.) to produce a new dataset with modified properties to improve model generalization using ground truth. The methods and systems described herein are not limited to training AI models based on patients who have been previously treated. For instance, instead of previously treated patients, the training dataset may include data associated with any set of participants (not patients) who are willing to be monitored for the purposes of generating the training dataset. Therefore, participants in a study who are not being treated can be connected to one or more electronic sensors where the analytics server includes data collected from the sensors within the training dataset.

Training the AI models 111 may be performed, for example, by analyzing historic patient data (e.g., patient's movements and their corresponding medical images and breathing patters). For instance, the training dataset may include 100 patients and their breathing data collected via respiratory sensors. The training data may also include corresponding medical images associated with the patient. Each medical image may include a timestamp that corresponds to a time stamp of the patient's breathing cycles. This raw information may be converted into machine-readable objects using the processes described herein, and associated with the ground-truth failure information (if applicable), which can operate as a label. Inputs to the models 111 include a set of machine-readable objects generated by the analytics server (received from the sensor 163). Model outputs may include a confidence score indicating a likelihood of a particular structure's location.

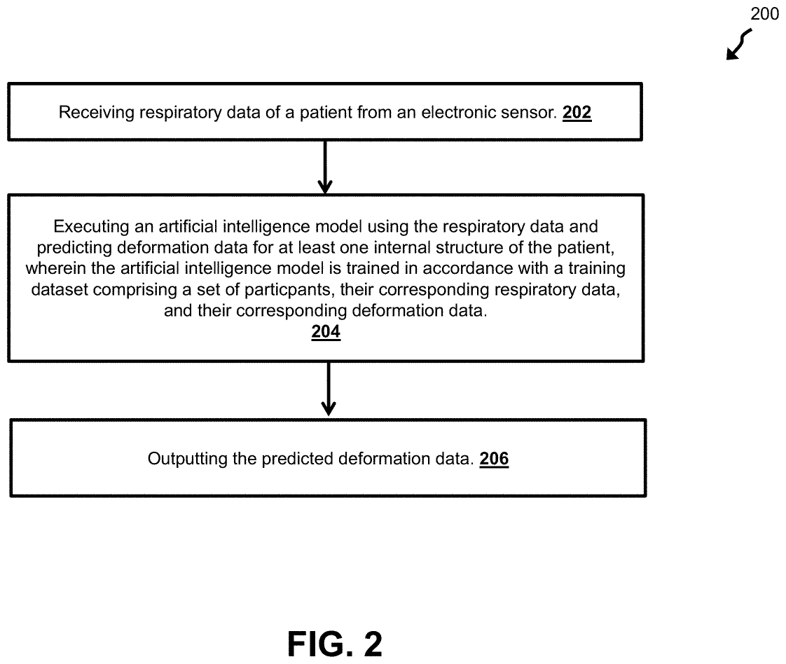

Referring to FIG. 2, depicted is an example data flow diagram 200 that shows how an AI model can be trained and executed to predict a patient attribute, in accordance with an embodiment. The method 200 may include steps 202-206. However, other embodiments may include additional or alternative steps or may omit one or more steps altogether. The method 200 is described as being executed by a server, such as the analytics server described in FIG. 1. However, one or more steps of method 200 may be executed by any number of computing devices operating in the distributed computing system described in FIG. 1. For instance, one or more computing devices may locally perform part or all of the steps described in FIG. 2.

Conventional methods of motion modeling obtain various images of a patient's internal structures while the patient is breathing and bin those images in accordance with the patient's respiratory pattern. Conventional methods then analyze the patient's internal structures (e.g., PTV or GTV) in accordance with the binned images to identify the location of a tumor and/or how the tumor moves as the patient moves (e.g., breathes). Conventional methods then assume that the patient continues breathing the same manner and predict the location of the tumor during treatment accordingly. This method is error-prone. In order to rectify conventional methods' inefficiency and inaccuracy, some medical professionals constraint the patient (e.g., abdominal binder, using ventilation apparatus forcing the patient to breathe in a certain pattern, or audio/visual coaching of the patient to breath regularly) in order to limit the tumor's movement. However, this is highly undesirable, as it restricts the patient's comfort during treatment. In another example, some other methods require additional kV imaging, which is also undesirable because the patient is receiving a higher dose.

Using the method 200, an AI model can be trained in accordance with a training dataset to predict how a patient breathes and consequently how the patient's respiratory data affects the patient's movement of internal structures. At implementation time, using the method 200, the AI model may be executed to predict how a patient's internal structures are deforming based on the patient's projected respiratory data. Moreover, unlike conventional methods, the method 200 allows for an AI model that can be executed using minimal patient data (e.g., surrogate signal and an initial medical image of the patient).

### Training of the AI Model

Before executing the AI model, the analytics server may first train the AI model and ensure its accuracy. The analytics server may train the AI model using a training dataset comprising two sets of data associated with a cohort of patients (or participants in a clinical trial).

First, the training dataset may include respiratory data associated with a set of patients and/or participants. Specifically, the training dataset may include data received from a respiratory sensor associated with each patient. This data is also referred to herein as the surrogate signal. The respiratory sensor may detect each patient's breathing patterns and movements (e.g., chest position and movement). The sensor may (or sometimes a separate processor or computing device may) transmit surrogate signals to a processor (e.g., analytics server) that records the surrogate signal and further analyzes it. The surrogate signal can be used to identify a respiratory cycle associated with each patient and a corresponding patient movement. In operation, in order to generate the training dataset, participants and patients may be asked to wear a respiratory sensor (or sometimes consent to being monitored via an optical or 3D surfacing mechanism). As a result, the respiratory sensor may generate respiration data (or surrogate signal) associated with each patient (e.g., respiratory cycle, chest position and movement, and the like).

Second, the training dataset may include structure deformation data associated with the set of patients. For instance, the training dataset may include medical images associated with the set of patients. The medical images may be periodically obtained while the patients are breathing and/or being treated. Each image may be taken from a particular anatomical region of the patient. For instance, in operation and in order to prepare a training dataset, medical images of patients and participants are periodically taken. Each image may include a timestamp that can be used to identify corresponding respiratory data associated with the medical image.

In an embodiment, the training dataset may include medical images (e.g., CT or 4DCT) depicting the patient's internal organs and corresponding sensor data identifying the patient's respiratory data (e.g., respiratory cycle). The training dataset may not only rely on external data and the patient's deformation. For instance, when preparing the training dataset with regard to patients being treated, the analytics server may also include kV projections within the training dataset. In some embodiments, the training dataset may also include MR and/or time-resolved MR data associated with the patient.

The training dataset may also include additional data associated with the patients (other than the surrogate signal or medical images). For instance, the AI model may consider each patient's demographic information and/or other biological markers (e.g., age, weight, or BMI). As a result, the model may also consider the patient's attributes when considering and relating how the patient's internal structures are deforming/moving and/or how the patient is breathing. In operation, some patients may lose weight during (and a result of) the treatment. Therefore, their deformation and/or the respiratory cycle may slightly change because of their weight loss. For instance, the tumor may shrink under treatment, which then could lead to varying motion patterns, even if the external surrogate remain unchanged.

The training dataset may also include each patient's medical history, such that the AI model may connect any corresponding data to how the patient's internal structures have deformed or are deforming. For instance, the training dataset may include whether a patient suffers from chronic obstructive pulmonary disease (COPD) or sleep apnea. If so, the training dataset may also include a value associated with the patient's COPD or sleep apnea's severity. In another example, the training dataset may include an indication of whether a patient is a smoker. In another example, a patient may suffer from a disease (e.g., cancer). The training dataset may include an indication of the disease and a corresponding stage of the patient's disease.

The patient's medical information may indicate how the patient breathes, and as a result, how the patient's structures deform/move. For instance, the model may learn that patients with COPD breathe faster than patients without COPD. As a result, patients with COPD may have different deformation data and may have internal structures that deform at a different rate.

When reviewed in totality, the training dataset may include information that could indicate how each patient's breathing affects their internal organs and structures. Specifically, the training dataset may indicate how breathing deforms or moves one or more internal structures of each patient. In operation, a set of patients may be asked to normally breathe while a chest sensor is monitoring/recording their respiratory data (e.g., chest movement and respiratory cycle data). Moreover, a medical imaging apparatus is used to periodically capture images depicting how the patient's internal organs are moving and deforming. When reviewed together, each image can be analyzed in view of its timestamp and a corresponding respiratory cycle of the patient.

The analytics server may then aggregate various datasets that are associated with the set of patients and include the aggregated datasets within the training dataset. Using the training dataset, the analytics server may train one or more AI models discussed herein. In various embodiments, the AI model may use one or more deep learning engines to perform automatic segmentation of images received and/or to correlate the data within the training dataset, such that they uncover patterns connecting how a patient breathes and how the patient's breathing/movement deforms or moves their organs or internal structures.

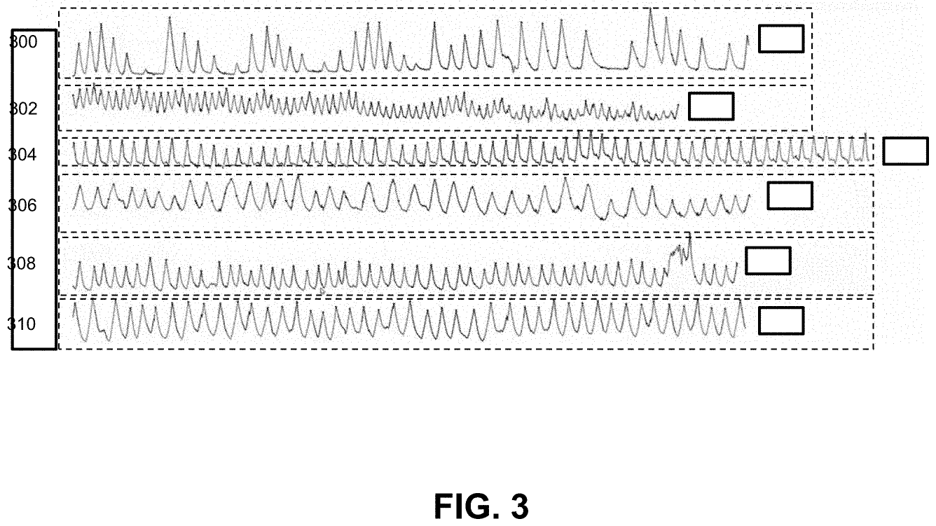

The AI model may first analyze the surrogate signal data and determine a respiratory cycle associated with each patient within the training dataset. As depicted in FIG. 3, the AI model may identify that different patients have different respiratory attributes and cycles. Each respiratory cycle depicts two phases (inspiration or inhalation and expiration or exhalation). For instance, each row 300-310 depicts respiratory data associated with a different patient. As depicted, different patients have different respiratory attributes (e.g., different patients breathe at different speeds and have different breath volumes). Furthermore, patients exhibit a change in their respiratory cycles over time (e.g., during treatment). For instance, some patients may relax as they acclimate to the radiotherapy environment and may change their breathing patterns (e.g., breathe slowly). The AI model may also learn that there are some common elements across the respiratory patterns (e.g., there may be a periodicity detected). For instance, the AI model may learn the common respiratory phases and/or how different patients change their respiratory pattern over time. As a result, may learn how to predict a patient's respiratory data at a given time.

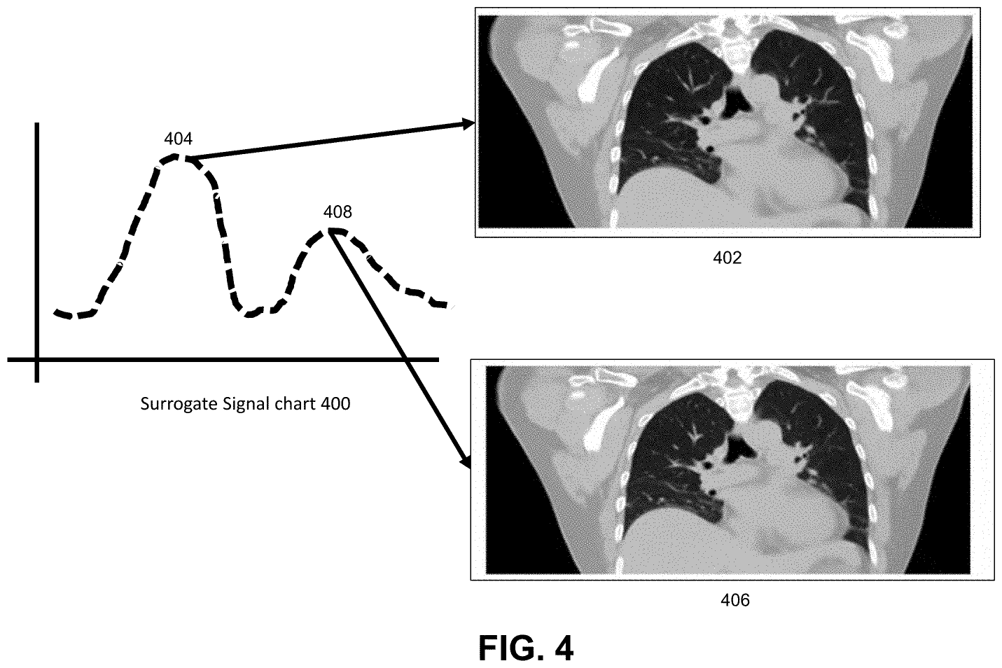

Using various machine-learning techniques, the model may identify how each patient (given their attributes) breathes. Moreover, the AI model may also ingest deformation data (e.g., medical images) and connect each deformation to its corresponding respiratory data/cycle. FIG. 4 depicts a correlation between a patient's internal structures and a corresponding surrogate signal. The surrogate signal chart 400 depicts a particular patient's surrogate signal that corresponds to their respiratory data captured using a respiratory sensor. The AI model may determine that the medical image 402 corresponds to the point 404 within the surrogate signal chart 400 and the medical image 406 corresponds to the point 408 within the surrogate signal chart 400. As depicted via the medical images 402 and 406, this particular patient's internal structures slightly move and deform based on the patient's breathing (as indicated by different points within the surrogate signal chart 400).

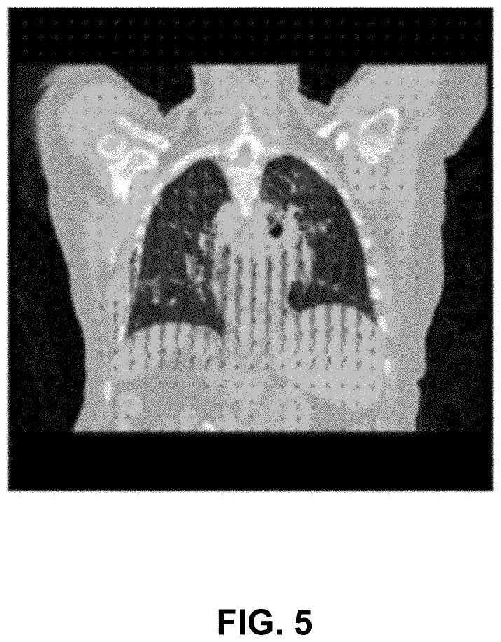

The AI model may learn how the internal structures deform or move. Utilizing a method, such as a conditional variational auto-encoder (conditional VAE), the AI model may identify how one or more structures would deform based on the patient's respiratory data. The model may vectorize the medical images received and prepare a vectorized location for each point within the medical images received, as depicted in FIG. 5. The AI model may then relate the vector to the surrogate signal. The AI may, after being properly train, predict a vector corresponding to how each point within a medical image would move/deform as the patient breathes. This predicted vector is also referred to herein as the deformation vector.

The AI model may also analyze the surrogate signal and identify a corresponding latent signal that corresponds to the surrogate signal. The AI model may use the latent signal to reconstruct a medical image associated with the patient's movement (even in cases without having the surrogate signal). The AI model may train itself based on a detected difference between timestamped medical images of a patient in light of the patient's respiratory data.

In the unsupervised learning method, the surrogate signal may not always be known to the AI model (as opposed to supervised learning methods in which the surrogate is known and may be labeled as the ground truth). In some embodiments, the AI model may predict the surrogate signal itself. For instance, the AI model may predict how the patient may be breathing during the treatment. Because some patients change their respiratory patterns (e.g., some patients relax and breathe more normally as the treatment progresses), the predicted respiratory data may be used instead of using the patient's initial respiratory cycle.

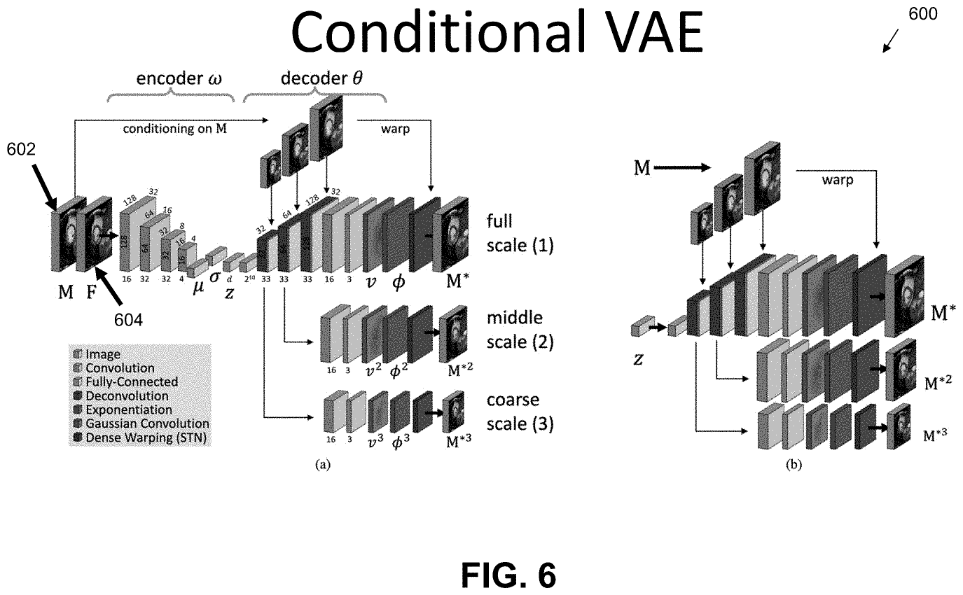

The AI model may analyze the images (including how the internal structures are moving) and their corresponding respiratory data to train itself, such that the trained AI model can ingest a surrogate signal and an initial medical image of a new patient and predict how the new patient's internal structures would move and deform. The AI model may use a variety of methods to uncover hidden patterns, such as using deep learning methods. The AI model may use an unsupervised or semi-supervised method in which moving images are automatically analyzed and deformations are highlighted. For instance, when the AI model receives a moving medical image (4DCT), the AI model may change the moving medical image into a fixed medical image, and identify various deformations and differences between the images. As depicted in FIG. 6, the AI model may use a conditional VAE method 600 in which the AI model ingests a moving medical image 602. The goal of the AI model can be defined as transforming the moving medical image 602 to one or more fixed images 604. However, the model (at this point within the training) may not ingest data identifying how various internal structures are deforming within the moving medical image 602. That is, the AI model may not receive data labeling how structures have moved or deformed (e.g., supervised training). This is mainly because the analytics server may utilize an unsupervised method in which the training is not limited to known technologies and known limitations of deformation vectors of different deformable image registration approaches. The AI model may compute a difference between the images and test its identification of the difference by comparing pixel intensity values (e.g., Hounsfield unit (HU) for CT/CBCT) between the different medical images. Specifically, each image may be divided into different segments (e.g., pixels or a collection of pixels) and pixel intensity values of corresponding segments may be compared to determine how internal structures have moved.

The AI model may test its own accuracy and recalibrate itself accordingly. For instance, the AI model may monitor the patient during treatment and determine whether the patient's actual internal structure locations match (within a tolerable threshold) with the patient's predicted structure locations. For instance, one or more medical imaging devices may periodically provide an image of the patient's internal structures and the captured image (actual) may be compared with the patient's predicted image. In another example, the analytics server may compare Kv imaging captured as a result of the patient's treatment with simulated images/data predicted/simulated by the model and validate the model's accuracy.

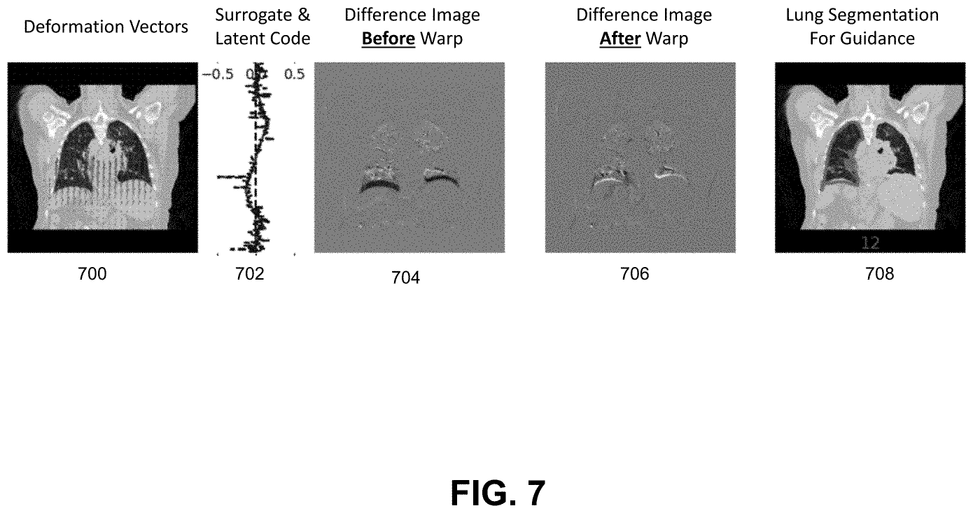

The AI model may also be trained to generate a predicted deformation medical image for the patient. For instance, the AI model can be used to generate a moving or fixed medical image representing how the patient's internal structures will move/deform. FIG. 7 depicts medical images predicting how a patient's lungs will deform. Using the methods and systems discussed herein, the AI model may predict the deformation vectors depicted in the medical image 700. The AI model may also account for the surrogate signal and may learn from the surrogate signal (e.g., learn how to replicate the signal, which is referred to as the latent signal or latent code), as depicted in the chart 702. The AI model may replace the surrogate signal received from the respiratory sensor with the latent signal to predict how an internal structure moves/deforms. As depicted, the latent and surrogate signals closely resemble each other.

The AI model may simulate a medical image to convey the predicted deformation data. For instance, the AI model may then generate the image 708 that identifies a projected deformation of the patient's lungs. The AI model may also use various segmentation protocols to segment one or more structures within a medical image. For instance, the image 708 can be segmented to highlight the position of the patient's lungs. The AI model may also utilize image warping to identify a difference/deformation (e.g., digitally manipulating an image) as depicted by images 704 and 706. In some embodiments, the analytics server may use a warping protocol to warp the images to a common reference/atlas.

Fitting and training AI models for individual patients (e.g., some conventional methods) does not utilize data account for the dynamic of respiration or the psychology of respiration. For instance, some patients may drastically change their breathing pattern once they are more relaxed or more acclimated to the treatment (e.g., muscle relaxation or breathing drift). In contrast, the AI model trained using the methods and systems discussed herein accounts for how patients breathe during their treatment.

In some configurations, the analytics server may pre-train or partially train the AI model. For instance, the analytics server may train the AI model based on a set of cohort patients. Then, the analytics server may train (fine-tune) the AI model using a particular patient's specific data. For instance, when the AI model is pre-trained, the analytics server may fine-tune the AI model and customize it to a particular patient by feeding information (e.g., respiratory data and medical images) of the patient. This allows for customizing the AI model without risking overfitting.

Using a cohort of patients to train the AI model allows the AI model to learn various attributes common among the set of patients, such as sliding interfaces or rigidity of bones. The AI model may then fine tune these learnings for a particular patient.

Execution and Implementation of the AI model

During training, the analytics server may iteratively produce new predicted results (recommendations) based on the training dataset (e.g., for each patient and their corresponding data within the dataset). If the predicted results do not match the real outcome, the analytics server continues the training unless and until the computer-generated recommendation satisfies one or more accuracy thresholds and is within acceptable ranges. For instance, the analytics server may segment the training dataset into three groups (i.e., training, validation, and test). The analytics server may train the AI model based on the first group (training). The analytics server may then execute the (at least partially) trained AI model to predict results for the second group of data (validation). The analytics server then verifies whether the prediction is correct. Using the above-described method, the analytics server may evaluate whether the AI model is properly trained. The analytics server may continuously train and improve the AI model using this method. The analytics server may then gauge the AI model's accuracy (e.g., area under the curve, precision, and recall) using the remaining data points within the training dataset (test).

After the model is trained, the AI model can predict deformation vectors. The deformation vectors identify how each point within an image will move/deform.

Referring back to FIG. 2, at 202, the analytics server may receive respiratory data of a patient from an electronic sensor. The analytics server may be in communication with one or more sensors configured to monitor a patient's movements. The electronic sensor may identify a respiratory rate for the patient by counting the number of breaths via counting how many times the patient's chest rises. In one example, the electronic sensor may be a wearable (e.g., chest strap or a patch over the chest) respiratory monitoring system that monitors the respiratory patterns of a patient. The electronic sensor may detect small changes in a patient's breathing pattern, chest position, tidal volume and/or other vital signs. In another example, a fiber-optic breath rate sensor can be used for monitoring the patient. In yet another example, various 3D surfacing methods may be used to determine how the patient is breathing. Additionally, the analytics server may retrieve one or more medical images (e.g., CT or 4DCT) of the patient.

At 204, Executing an artificial intelligence model using the respiratory data and predicting deformation data for at least one internal structure of the patient, wherein the artificial intelligence model is trained in accordance with a training dataset comprising a set of participants (e.g., previously treated patients or participants of a clinical trial), their corresponding respiratory data, and their corresponding deformation data. As used herein, deformation data, refers to any data predicted by the AI model. Non-limiting examples of deformation data may include any data (e.g., deformation vectors, numbers, and simulated medical images) that convey how one or more internal structures would move or deform at a given time.

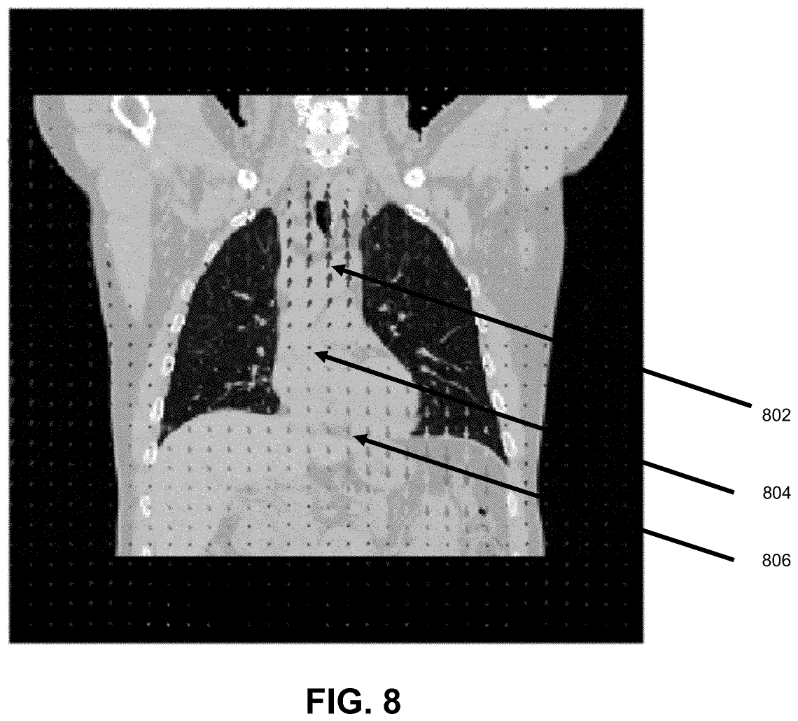

The analytics server may execute the AI model discussed herein using the data received in step 202. Additionally, the analytics server may receive an initial medical image of the patient. The AI model may be trained in accordance with the methods and systems discussed herein. Because of the execution, the AI model may predict deformation data associated with one or more organs or internal structures of the patient. Specifically, the AI model may predict deformation vectors indicating how each point within a medical image of the patient will move/deform. The deformation vectors may indicate a distance and direction that each point within the medical image will move. For instance, as depicted in deformation vectors 800 (FIG. 8), vector 802 indicates that its corresponding point within the medical image will move upwards (e.g., by 1 millimeter) and the vector 804 indicates that its corresponding location will not move. In contrast, the vector 806 indicates that its corresponding location will move downwards (e.g., 0.5 millimeter). Using the deformation vectors, the analytics server may predict a location and orientation of one or more internal strictures of the patient.

At step 206, the analytics server may output the data predicted by the AI model (deformation data). The analytics server may output the deformation data in multiple ways. In one embodiment, the analytics server may output the deformation vectors. For instance, a GUI accessed by a medical professional may display an image similar to the depicted in FIG. 8 where different deformation vectors and their corresponding magnitude and direction are depicted.

In another example, the analytics server may use the AI model to generate a moving or fixed medical image that depicts how the patient's internal structure would move/deform. For instance, a GUI accessed by a medical professional may display a projected 4DCT of the patient that depicts how the patient's internal structures are going to move/deform.

In another example, the analytics server may revise one or more attributes of the patient's radiotherapy treatment using the data predicted by the AI model. For instance, the analytics server may revise an attribute of a multi-leaf collimator (MLC), move the couch, or pause the beam, or a combination of any of these examples. Specifically, in conjunction with one or more other software solutions, the analytics server may revise an opening of the MLC, such that radiation dissemination is directed towards the projected location of a PTV (e.g., projected using the AI model). In this way, the analytics server provides a dynamic MLC correction method where the MLC openning can be revised in real-time or near real-time.

Effectively, the analytics server may enable gating of the beam to match the motion of the patient's tumor. Because the analytics server can predict/estimate the tumor location, the analytics server may control one or more attributes of the radiotherapy machine. For instance, the analytics server may control (e.g., review and revise) the MLC opening, timing, and/or the dose rate.

In another example, the analytics server may transmit the data predicted via the AI model to a downstream software solution. For instance, using the results of execution of the AI model can be transmitted to a dose calculation software solution. In another example, the analytics server may transmit the deformation data to a downstream tissue tracking application.

In a non-limiting example, when a patient is positioned on a bed of a radiotherapy machine (before receiving treatment), the patient is asked to wear a respiratory sensor. The analytics server retrieves the surrogate signal received from the respiratory signal. Also, the analytics server retrieves an initial medical image (CT) of the patient. Using the surrogate signal and the medical image, the analytics server execute an AI model that has been trained using the methods and systems discussed herein. As a result, the AI model generates deformation vectors and simulates a new medical image that predicts how the patient's internal structure would move and deform. The simulated medical image (e.g., simulated 4DCT) is displayed on a GUI accessed by a medical professional treating the patient. When the patient's treatment starts, the analytics server also revises MLC openings of the radiotherapy machine in real time in accordance with the AI model's predicted results. The analytics server also communicates with a tissue tracking and dose calculation software solution and transmits the predicted results to said software solutions.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium.

The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What we claim is:

1. A method comprising:
receiving, by a processor, respiratory data of a patient from an electronic sensor;
executing, by the processor, an artificial intelligence model using the respiratory data and predicting deformation data for at least one internal structure of the patient,
wherein the artificial intelligence model is trained in accordance with a training
dataset comprising a set of participants, their corresponding respiratory data, and
their corresponding deformation data; and
outputting, by the processor, the predicted deformation data.

2. The method of claim 1, further comprising:
receiving, by the processor, a medical image of the patient, wherein the processor executes the artificial intelligence model using the medical image.

3. The method of claim 1, wherein the respiratory data received from the electronic sensor is at least one of a chest position, chest movement, or respiratory cycle data of the patient.

4. The method of claim 1, wherein the deformation data corresponds to a movement of at least one internal structure of the patient.

5. The method of claim 1, further comprising:
adjusting, by the processor, at least one attribute of a radiotherapy machine in accordance with the predicted deformation data.

6. The method of claim 5, wherein the at least one attribute corresponds to at least one of a multi-leaf collimator opening, pausing a beam, or moving a couch.

7. The method of claim 1, wherein outputting the predicted deformation data corresponds to a simulated medical image depicting an anatomical region of the patient.

8. The method of claim 1, wherein outputting the predicted deformation data corresponds to transmitting the predicted deformation data to a dose calculation software solution or a tissue tracking software solution.

9. The method of claim 1, wherein the artificial intelligence model generates predicted respiratory data associated with the patient, the predicted respiratory data comprising at least one of a chest movement or an attribute of a respiratory cycle.

10. The method of claim 1, wherein the electronic sensor is a wearable respiratory sensor or an optical respiratory sensor.

11. A computer system:
a server comprising a processor and a non-transitory computer-readable medium containing instructions that when executed by the processor causes the processor to perform operations comprising:
receiving respiratory data of a patient from an electronic sensor;
executing an artificial intelligence model using the respiratory data and predicting deformation data for at least one internal structure of the patient, wherein the artificial intelligence model is trained in accordance with a training dataset comprising a set of participants, their corresponding respiratory data, and their corresponding deformation data; and
outputting the predicted deformation data.

12. The computer system of claim 11, wherein the instructions further cause the processor to receive a medical image of the patient, wherein the processor executes the artificial intelligence model using the medical image.

13. The computer system of claim 11, wherein the respiratory data received from the electronic sensor is at least one of a chest position, chest movement, or respiratory cycle data of the patient.

14. The computer system of claim 11, wherein the deformation data corresponds to a movement of at least one internal structure of the patient.

15. The computer system of claim 11, wherein the instructions further cause the processor to adjust at least one attribute of a radiotherapy machine in accordance with the predicted deformation data.

16. The computer system of claim 15, wherein the at least one attribute corresponds to at least one of a multi-leaf collimator opening, pausing a beam, or moving a couch.

17. The computer system of claim 11, wherein outputting the predicted deformation data corresponds to a simulated medical image depicting an anatomical region of the patient.

18. The computer system of claim 11, wherein outputting the predicted deformation data corresponds to transmitting the predicted deformation data to a dose calculation software solution or a tissue tracking software solution.

19. The computer system of claim 11, wherein the artificial intelligence model generates predicted respiratory data associated with the patient, the predicted respiratory data comprising at least one of a chest movement or an attribute of a respiratory cycle.

20. The computer system of claim 11, wherein the electronic sensor is a wearable respiratory sensor or an optical respiratory sensor.
